(19) United States
(12) Patent Application Publication
(10) Pub. No.: US 2006/0195167 A1
(43) Pub. Date: Aug. 31, 2006

(54) Electrostimulating system

(76) Inventor: Andrea Zanella, MIRANDOLA (IT)

Correspondence Address: NIXON & VANDERHYE, PC, 901 NORTH GLEBE ROAD, 11TH FLOOR, ARLINGTON, VA, 22203, US

(21) Appl. No.: 10/550,601

(22) PCT Filed: Mar. 26, 2004
(86) PCT No.: PCT/EP04/03270

§ 371 Date: Oct. 24, 2005

(30) Foreign Application Priority Data
Mar. 28, 2003 (IT) MO2003 A 000089

(51) Int. Cl. A61F 7/00

(52) U.S. Cl. 607/96

(57) ABSTRACT

An electrostimulating apparatus that generates a relaxing sequence suitable for stimulating the striated or vasoactive muscle fibre for the activation of the microcirculation, based on three fundamental parameters: the width of the electric stimulation: the frequency of said stimulation and the time intervals wherein a plurality of width/frequency combinations follows.

The invention refers to an electrostimulating system comprising means for producing an electric stimulation that consists of bioactive neuromodulation of the neurovegetative system, of the striated-muscle system, of the smooth muscle and of the mixed nervous structure, particularly suitable for producing inter alia phenomena of muscular contraction and relaxation by means of emulation of the action of the nerve fibre that innerves a skeletal muscle or of the neuroceptors of the sympathetic system that interact with the smooth muscle of the vessels.

Equally, depending on the type of electric stimulation and on the configuration parameters, a consequent induced bioactive neuromodulation can be generated that is suitable for producing vasoactive phenomena in the microcirculaton and in the macrocirculation, which are in turn mediated by phenomena connected with the direct stimulation of the smooth muscle and by essentially catecholamine energy phenomena by means of stimulation of the postsynaptic receptors.

The system thus produces stimulation sequences that induce reproducible and constant neurophysiological responses; in particular, but not restricted thereto, the sequences of activation of the microcirculation (ATMC) and relaxation of the muscle fibre (DCTR) are able to stimulate different functional contingents, including but not limited to the striated muscle, the smooth muscle and the peripheral mixed nerve.

The stimulation sequences are assembled on three fundamental parameters: the width of the stimulus, the frequency of the stimulus and the time wherein different combinations of width/frequency follow each other. The general operating model reflects the digital-analogue transduction that occurs in nervous transmission.

WO 02/09809 discloses an apparatus for the treatment of muscular, tendinous and vascular pathologies by means of which a series of electric pulses lasting from 10 to 40 microsecs are applied to a patient and at variable intensity, depending on the impedance and conductance of the tissue subjected to stimulation, typically from 100 to 170 microampere. These electric pulses are able to produce a relaxing, anti-inflammatory and vasoactive effect. Such levels of current and the connected level of energy transferred, below 5 microjoules, cannot create polarisation or ionisation of metallic structures and are therefore absolutely compatible with the presence in the stimulated organism of, for example, metal prostheses, or of intrauterine-coil contraceptive devices and cardiostimulators or implanted defibrillators (pacemakers).

U.S. Pat. No. 5,725,563 discloses a method and a system of adrenergic stimulation of the sympathetic nervous system relative to the circulation of the patient wherein electric pulses are generated and simultaneously impedance of the cytoplasm contained in the space between the stimulation electrodes is measured. In this case, the specific effects of the disclosed system are cited, namely the vasoconstriction that is a consequence of activation of the alphaadrenergic postsynaptic receptors that modify the venous tone, thereby producing vasoconstriction and consequent vascular and lymphatic drainage. In this case, to obtain this specific effect, stimulations are proposed in a range of frequencies absolutely below 2 Hz and preferably of 1.75 Hz with currents below 350 microAmperes and preferably below 250 microAmperes with energy transfer around 10 microjoule. In particular, the pulses generated by the above-mentioned stimulator are subordinated to the measurement of impedance so as to vary the width of the pulse in function thereof.

However, this system produces only the effect of a “peristaltic pump” due to the periodical “vasoconstriction” and subsequent “long” period of “relaxation” and is obtained by means of the delivery of very low frequency pulses (<2 Hz) to the smooth muscles of the vessel. However, in addition to being limiting and requiring careful measuring of impedance, it produces limited effects and requires stimulations that are extremely prolonged over time to obtain visible and effective effects.

On the contrary, this invention also solves all the problems that beset the prior art and significantly increases the disclosed positive effects, having a direct action on postsynaptic activity, it produces direct effects on synapses or the motor plate of the skeletal muscle involved.

The invention provides a combination of: an electrostimulating apparatus for applying electrical stimuli to biological tissues; heat exchanging means, arranged to exchange heat with said tissues.

Advantageously, the apparatus and the method provided by the invention exploit the principle of achieving significant bioreaction variations.

The invention may be better understood with reference to the attached drawings that illustrate certain embodiments by way of non-limiting example, wherein:

FIG. 1 shows a Cartesian graph of time/intensity of current, disclosing the intensity and time thresholds;

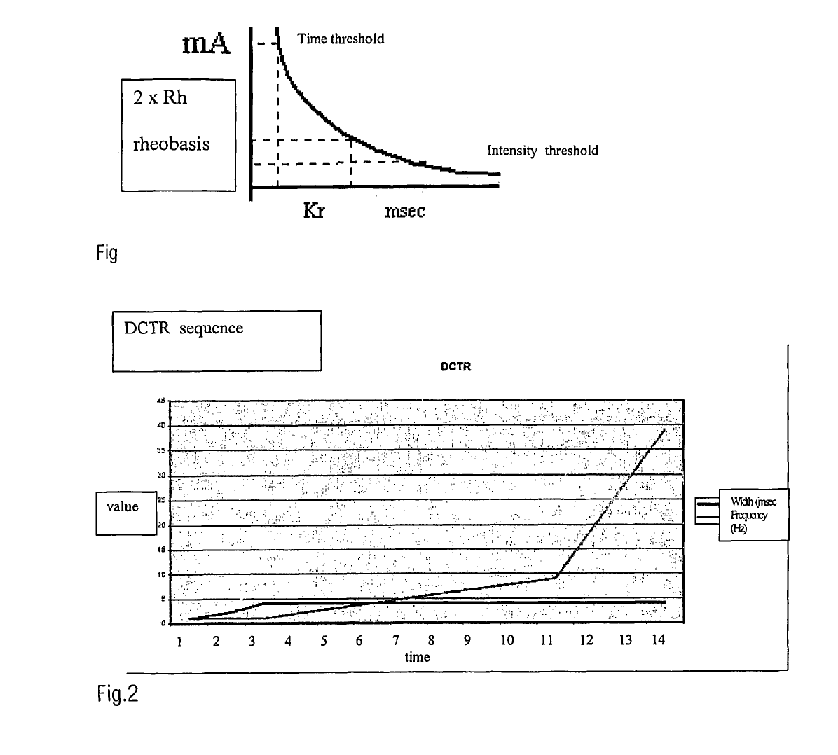

FIG. 2 shows a graph illustrating a relaxing sequence, or DCTR sequence, according to the invention;

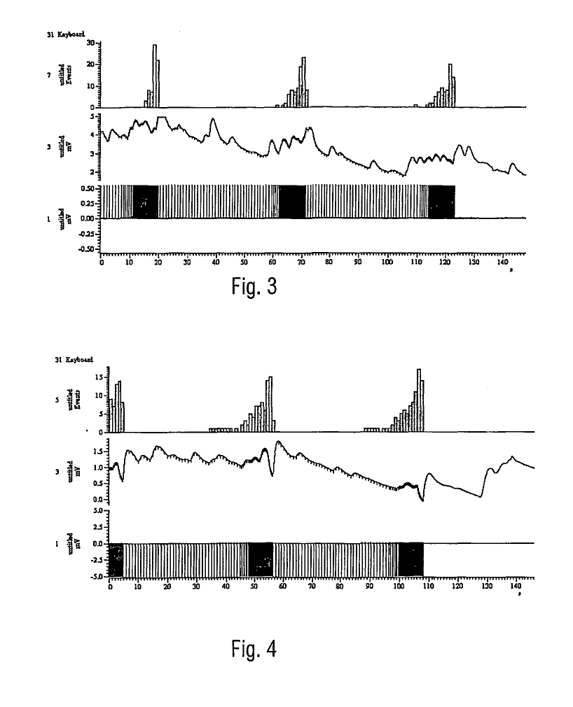

FIG. 3 shows a DCTR sequence plot, carried out on a healthy subject;

FIG. 4 shows a plot like the one in FIG. 3, but carried out on a further healthy subject;

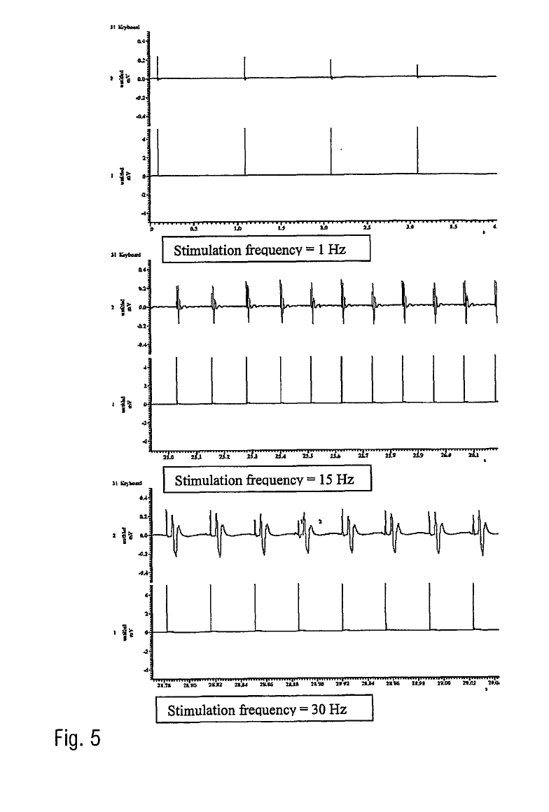

FIG. 5 shows three surface electromyograms, with stimulation frequencies of 1, 15 and 30 Hertz;

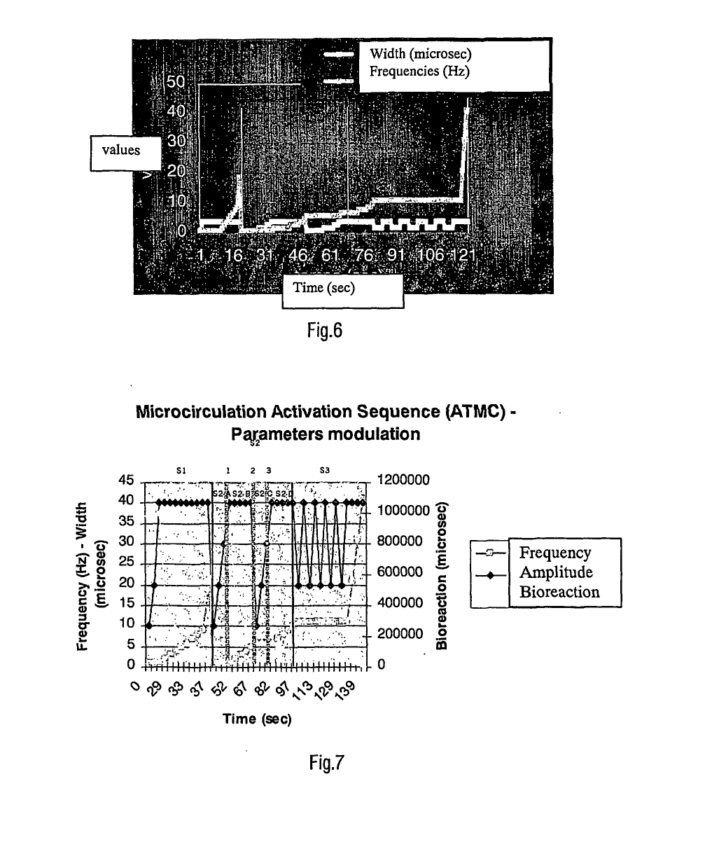

FIG. 6 shows a graph illustrating a reactivation sequence of the microcirculation, or ATMC sequence, according to the invention;

FIG. 7 shows a polygraph recorded during administration of an ATMC sequence to a healthy subject, in the presence of electric stimulation;

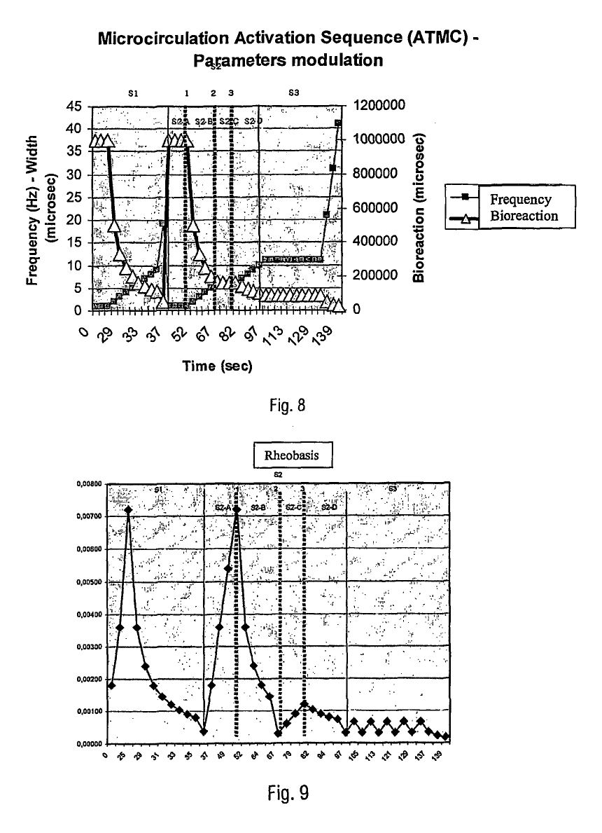

FIG. 8 shows a polygraph like the one in FIG. 7, but conducted in the absence of electric stimulation;

FIG. 9 shows a graph highlighting the discontinuous variation of the bioreaction obtained during administration of an ATMC sequence;

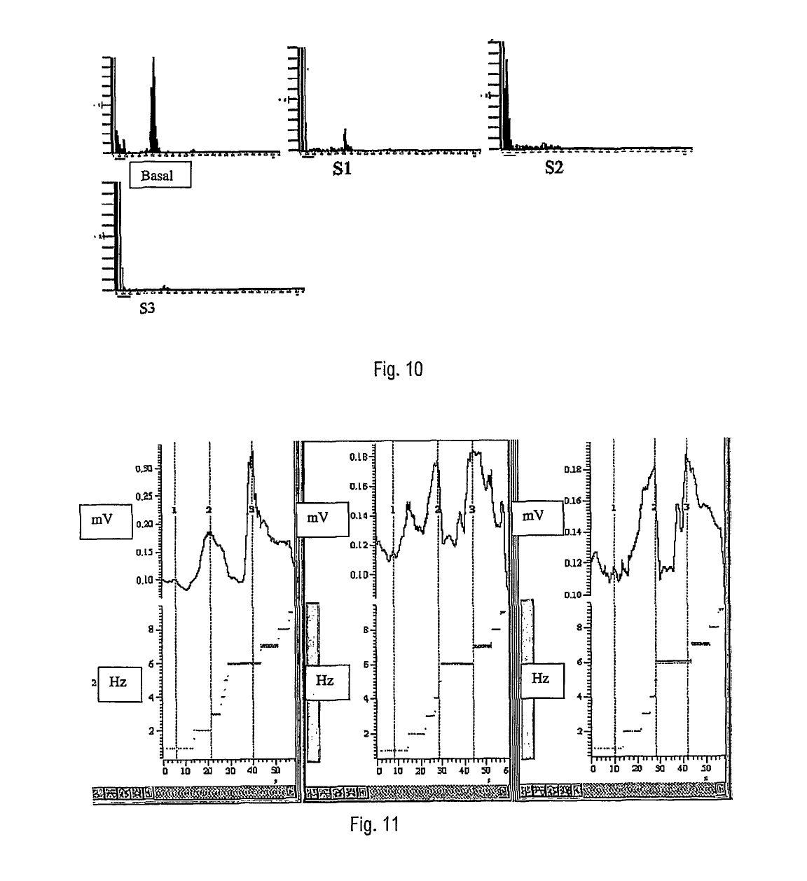

FIG. 10 shows graphic histograms of flow plots recorded in the presence and/or absence of ATMC sequences;

FIG. 11 shows flow variations recorded at the same time as the administration of an ATMC sequence like the one illustrated in FIG. 7;

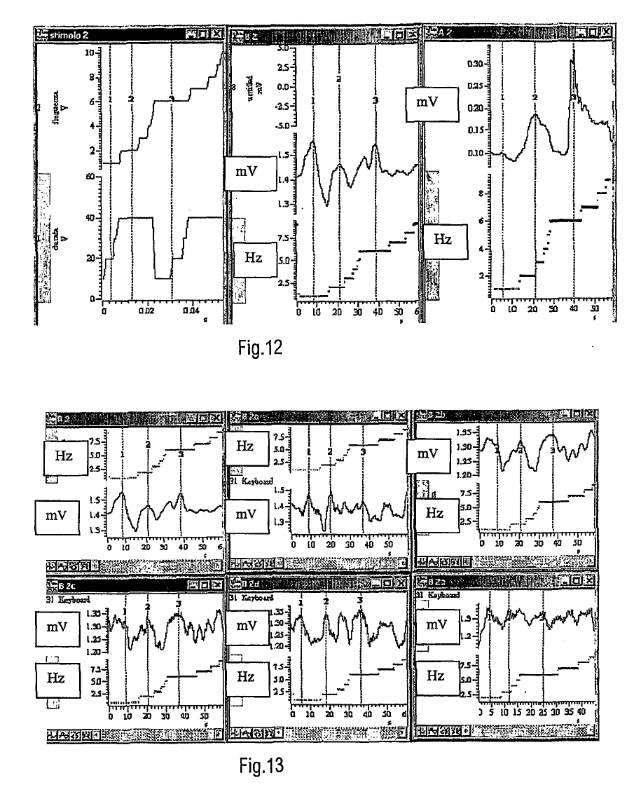

FIG. 12 shows flow variations similar to those in FIG. 11, but recorded during the administration of an ATMC sequence like the one illustrated in FIG. 8;

FIG. 13 shows further flow variations like those in FIG. 12;

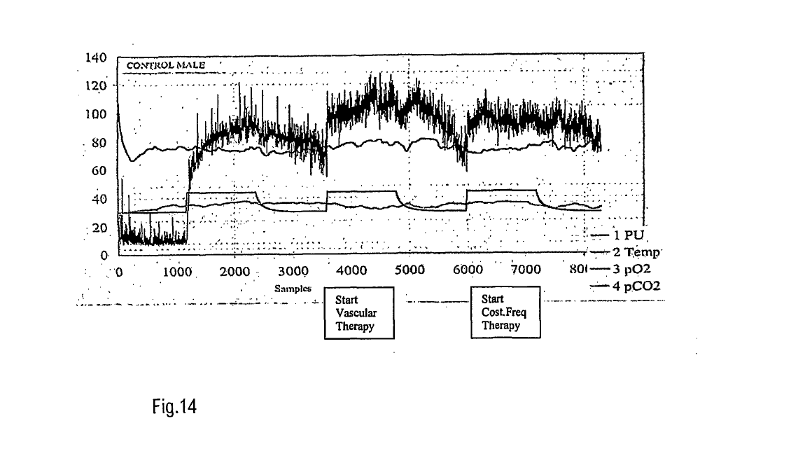

FIG. 14 illustrates a combination of an ATMC sequence with a thermal heating stimulus.

The nervous cell is responsible for the formation and transmission of the nervous pulses, which regulate the operation of the entire organism. This nervous cell is formed by a cell body or “soma” wherefrom branches lead: the “dendrites” along which the pulse has a centripetal direction (i.e. towards the cell body) and the “axon”, along which the pulses are mediated by the soma to the periphery, i.e. in a centrifugal direction. The pulses that do not arise from the soma of the cell are transmitted to the latter by other nervous cells or by specialised structures (receptors) or originate directly with the fibres, as in the case of free nerve ends responsible for collecting painful stimuli.

The pulse can travel towards the centre or vice versa. In the first case it is defined as being afferent and the result, analysed at the level of the Central Nervous System, is the acquisition of conscious information (sensitive stimulation) or unconscious information (e.g. automatic regulation of balance). The pulse that travels from the centre to the periphery is therefore defined as efferent and is able to cause the stimulation of the innerved organ or tissue.

The result of this may be muscular contraction, a glandular secretion, variations in cell metabolism, vasodilatation, vasoconstriction, and so on. Transmission of the pulse between the nerve fibres and the cells of a tissue occurs with the help of synapsis. The latter is terminal dilation (terminal button) of the axon that is in contact with the membrane of the cell to which the pulse is transmitted. A diminution of membrane potential in turn causes depolarisation that subsequently extends to the entire cell. The pulse that runs along the nerve fibre is merely the propagation of a depolarisation wave called action potential.

The nervous pulse may arise directly from the cell, but more often it originates from the stimulation of one of its parts, stimulated for example by pressure or a painful sensation.

The striated muscle fibre consists of thousands of myofibrils, consisting of two types of filamentous protein, that are arrayed in an alternating manner: the bigger the myosin the thinner the actin. The actin has light streaks defined as I bands, whereas with actin and myosin dark streaks known as A bands are created. The complex formed by an A band and by two adjacent semibands I is given the name “sarcomere”. Between two adjacent sarcomeres there exists a contact zone and a sarcoplasmic reticulum for the control of the contraction consisting of two different types of tubules: T tubules and longitudinal tubules.

Each muscle fibre receives pulses from the motor nerve fibre via the neuromuscular junction, which takes the name motor plate.

When the pulse arrives this causes depolarisation known as “plate potential” which generates action potential along the entire length of the muscle fibre, which causes it to contract. It is at this point opportune to recall the definition of the “chronaxy” and “rheobasis” parameters regarding the excitability characteristics of the nerve and muscle fibres. Chronaxy (Kr) is defined as the time (expressed in msec) required by a current intensity to reach a value that is twice the rheobasis (muscle sensitivity). Rheobasis (Rh) is in turn defined as the minimum (liminal) measurable current intensity required to excite a cell.

If the stimulating current is limited to a short time of the order of msec it will be observed that the shorter the width of the current is, the greater its intensity will have to be to reach the threshold. As shown in FIG. 1, by plotting the intensity-time curve two intensity and time thresholds are defined. The theoretical construction of the curve is achieved on the basis of the capacitive features of the axon membranes. The higher excitability is, the more concave the curve will be in relation to the axes because smaller products (i·t), i.e. smaller quantities of electricity will correspond to its points. When one wishes to determine the excitability of a nerve or muscle in vivo chronaxy is used. Chronaxy and rheobasis are in fact interconnected as characteristics of the nerve fibre. By means of “Lorenz stimulation with modulated frequency and amplitude” the excitation of the nerve fibres can be obtained by means of the summation effect of several subthreshold signals that are not able to excite the fibre, which however, by combining their effects together, are able at a certain point to excite the fibre. The summation effect, with the same produced pulse amplitude, will depend on the amplitude of the signal and on the bioreaction that is therefore connected to frequency, which in turn interact with the rheobasis-chronaxy ratio.

To demonstrate this behaviour, an analytical study of the physiological responses was conducted in combination with “Lorenz stimulation” by applying two different experimental procedures.

A first procedure is based on the use of a relaxing action sequence or DCTR, whose frequency and width characteristics are set out in FIG. 2.

The aim of the reported experiment is to prove the validity of the hypothesis that such a sequence, disclosed in WO 02/09809 and appropriately designed to have a relaxing effect on the muscle fibres, has a prevailing action on the activity of the skeletal muscle. Stimulation was achieved by measuring with sophisticated digital polygraph laboratory instruments with the possibility of sampling high-speed and high-frequency signals. The latter were recorded at the level of the short adductor muscle of the thumb and palm of the hand. For the short adductor muscle of the thumb a pair of plate electrodes (Ag+Cl−) was used through preamplification of the analogue signal at 5000 gains, passband 5 Hz-3 KHz. To the palm of the hand an electro-resistant transducer was applied comprising two surface electrodes, with 1:10 μohm preamplification.

The DCTR stimulation sequence was administered to two different healthy subjects. For each of them four polygraphs were recorded (as described previously), for three identical DCTR sequence cycles run consecutively. Two of the above polygraphs, obtained from different subjects, were illustrated in FIGS. 3 and 4. The stimulator electrodes were placed near the recording seats, along the route of the median nerve on the palmar surface of the wrist.

In both plots, carried out on healthy subjects, the median nerve was stimulated at the wrist with the DCTR sequence repeated three times, measuring on the short adductor muscle of the thumb of the thenar eminence with a transducer of skin impedance.

Each polygraph contains three plots separated into: top, middle and bottom.

The top plot shows the muscle responses obviously after discounting the stimulation artefacts, which responses are expressed in frequency histograms, whilst in the intermediate plot the skin conductance variations appear. In the bottom plot the stimulation sequence is shown, wherein the graphically “densest” part represents the rapid increase phases of the frequency.

As can be seen from the analysis of the DCTR sequence, the basic variation is the variation in the frequency of stimuli whereas widths remain constant at 40 microseconds.

In both polygraphs one notes the reproducible skin conductivity response (intermediate plot) in close temporal relationship, at about 500 msec latency, with the frequency increase phase of the stimulation. In both cases, the average conductance trend tends to fall. However, the absolutely original element and result of the disclosed invention consists of the close reproducibility of the responses regardless of the manner that they assume compared with the three phases of stimulation frequency.

This indicates that there is a direct dose-response relationship between the variability of the frequency of the electric stimuli which have a constant amplitude and are below the pain threshold and catecholaminergic vegetative efferents, inasmuch as skin conductance is directly influenced by local sweating, which is in turn carried, in the palm of the hand, by sympathetic innervation.

With regard to variation in skin conductance, some characteristics have emerged that are practically constant and independent of the subject subjected to stimulation and are disclosed below.

Above all, during the phase of rapid increase in stimulation frequency, a complex twin, triple or quadruple negative deflection phase occurs that is constant in each test during the three increase phases in both subjects and is therefore independent of the subjects themselves.

Again, the average trend of conductance under stimulation appeared to be indifferently ascending or descending in the different polygraphs. Characteristic trends and morphologies of the polyphase response belong to each subject.

Lastly, the overall duration of the polyphase response during the increase phase varies from 14 to 19 seconds; the greatest negative deflection is always the last of the complex and always occurs following the cessation of the incremental phase of the stimulus, with latency of approximately 1.5 sec. The negative components of the complex, which are variable between subjects and over the course of different measurements, always appear in relation to the first seconds of increase of the stimulation frequency.

In terms of the surface electromyogram, in both subjects and in all the measurements made, the same phenomena were ascertained, as described below.

During the preparatory stimulation, at a frequency of 1 Hz, there was no muscle response; during the increase phase composite motor unit potential was formed with increasingly shorter latency and increasingly higher amplitude until the formation of composite muscle action potential (cMAPSs) with minimum latency and maximum amplitude at the peak of the stimulation frequency.

The minimum appearance latencies of the cMAPSs correspond to the latencies that are detectable by means of electroneurography using standard methods. On the other hand, compared with the above-mentioned method of detection of the cMAPSs, the amplitudes are reduced by about 30%.

Each cMAP follows on from each stimulus and the isoelectric line of the plot returns after the cMAP to the value 0.

The top plot simply describes the production of composite motor potentials (cMAPs) in close temporal relation with the stimuli of the sequence. The inventive and original element consists of the fact that the first cMAPs appear only in the phase of increase of the frequency of the stimulation, according to a model that is absolutely analogous to the temporal recruitment of stimuli of the same amplitude, but placed in an increasing sequence over time (in a completely analogous manner to what occurs in the classical nerve-muscle physiological model).

The second phenomenon should also be pointed out, i.e. the one according to which, in addition to recruiting in frequency the number of cMAPs, the increase in stimulation determines the total amplitude of the cMAPS. This means that DCRT-type stimulation can perfectly emulate the action of a nerve fibre that innerves a skeletal muscle.

A second experimental procedure is based on the use of a reactivation sequence of the microcirculation, or ATMC, whose frequency and width characteristics are disclosed in the graph in FIG. 6.

This second procedure had the object of showing the validity of the hypothesis that an ATMC sequence, suitably designed to obtain the desired effect, has a prevalent action on the motility of the microcirculation, i.e. of the smooth sphincters of the arterioles and venules of the subcutaneous layer.

In this case, and for this object, stimulation was carried out by recording with a doppler flow laser-apparatus that is able to measure the degree of perfusion of the microcirculation, i.e. of the subcutaneous haematic flow, in addition to other correlated and synergic parameters, i.e.: O₂ saturation, CO₂ saturation and skin temperature.

To view the significant components of this sequence, with reference to FIGS. 5, 7, 8 and 9 the constitution of the ATMC sequence in three subsequences known as S1, S2, S3 is discussed below.

S1 and S3 are both characterized by a frequency increase phase, with distinct time modes, whilst S2 is mainly constituted for producing variability in the width of the different stimuli, in a gradually increasing range of frequencies but in such a way as to reduce the bioreaction until it is stabilised.

More in detail, during the S1 subsequence, a sequence that typically has a relaxing effect and which is very similar to the DCTR sequence disclosed above, different subphases are carried out wherein, after a first subphase with a 1-Hz frequency of mere adaptation, the frequency with a constant amplitude is gradually increased, thereby also gradually decreasing the bioreaction. Subsequently, the frequency is increased much more rapidly up to the target of 19 Hz.

Subsequently, the subsequence S2 is carried out, which in turn is subdivided into four parts, S2-A, S2-B, S2-C and S2-D. In this subsequence, after a phase wherein the amplitude is rapidly increased up to the instant 1 (S2-A), the frequency is made to gradually increase, and as a result the bioreaction rapidly falls to the instant 2 (S2-B). At this point the amplitude is reset, which will again increase at a constant frequency up to instant 3 (S2-C); the frequency will thereafter once again gradually increase at constant amplitude, as a result the bioreaction will also gradually fall to the instant 3 (S2-D).

In this way, the bioreaction is made to vary in a discontinuous manner, producing points of variation of the jump gradient, i.e. the points 1, 2 and 3.

To conduct the experiments, the sensor of the laser apparatus was placed on the extensor surface of the wrist (non-smooth skin). The stimulation electrodes were placed with the anode (stimulator) on the route of the radial nerve on the extensor surface of the third distal of the forearm and with the cathode placed near the proximal capitulum of the second phalanx. Furthermore, measuring electrodes of skin conductivity were positioned, in the same way as the first experimental procedure described above used to vary the effects of the DCTR sequence. The ATMC sequence was administered also in this case to two healthy subjects.

On the first a polygraph was first recorded during electric stimulation with an ATMC sequence and subsequently another polygraph of similar width was recorded but in absence of electric stimulation.

On the second subject two polygraphs were recorded, one of which compares responses during and after raising local skin temperature to 44° C. This thermal shock was induced by the instrument itself, whose laser probe in contact with the skin is provided with a thermistor able to heat the face of the probe in contact with the skin until a desired temperature is reached.

In this context it is important to stress that that was done because skin thermal stimulation is reported in the literature to be the maximum stimulation to obtain vasodilatation. Therefore in this case the intention is also to carry out a comparison.

Any stimulation carried out is made up of three basic identical sequences of the ATMC type.

The parameters that are most subject to variation are local flow, temperature and skin conductance, whereas oxygen and carbon-dioxide saturation do not show suggestive variations in relation to the sequence of the different stimulation phases. The analysis that is suggested by the detailed evaluation of the recorded plots enables the apparent synchronisation and desynchronisation of flow variation to be checked in relation to the incremental phases of the stimulation sequences. In fact, during the first subphase consisting of 30 seconds of constant stimulation at 1 Hz and at 40 microseconds of pure preparation (considerable ineffective stimulation), there is an increase in the average oscillation frequency of the flow signal by means of doppler laser, which instead enters at lower frequencies in a temporal relationship with the increase and decrease phases of the stimulation sequence.

In FIG. 10, the frequency spectra of the flow plot for each stimulation subsequence have been analysed by a Fourier transform in the field of frequencies, and compared with the spectrum over a period of recording without ATMC stimulation (base datum) and having a similar width (about 50 sec).

It can be noted that during the period without stimulation the oscillation frequencies are rather dispersed and prevalent on the 1-2 Hz band, i.e. the typical frequency of the heartbeat, whilst during the three stimulation subsequences frequencies are drastically synchronised on the 0-1 Hz range.

In detail, the response mode of the flow in relation to specific moments of the stimulation sequence is displayed. In the two subjects subjected to polygraph, the most constant flow variations could be observed during the subsequence S2.

In the plot recorded for subject 1 during the subsequence S2 and illustrated in FIG. 11, the bottom line indicated the frequency trend of stimulation, the top line indicated the virtually constant polyphase trend of the local subcutaneous flow variation.

In the plot recorded for subject 2 during the subsequence S2 and illustrated in FIG. 12, the flow line has a ‘peaks’ pattern whereas the line of the stimulation frequencies has a ‘steps’ pattern.

Although apparently random, the flow oscillation phases coincide perfectly with the different frequency variation phases of the stimulus.

The close correlation between the trend of the subsequence S2 and the flow response can be displayed through individuation of flow peaks that coincide with the instants 1, 2, 3 disclosed previously.

With reference to FIG. 13, at the points of flow peak a reversal occurs of the second derivative of the bioreaction and of the energy transferred to the tissue, and therefore of the determining chronaxy/rheobasis correlated therewith, in view of the characteristic of the phenomenon of temporal summation that occurs, i.e. a drastic jump variation of the first derivative thereof.

In practice, the system produces a sequence of vasodilatations and vasoconstrictions with sequential increases and decreases of the haematic flow of the microcirculation that produce a “pump” effect that is evidently produced by neuromodulation of the neurovegetative and of the sympathetic system, which influences vasoactivity through the smooth muscle of the smaller blood vessels (arterioles, capillary blood vessels).

During the subsequence S2 of the ATMC sequence, characterized by alternating variations of the rheobasis, a vasoactive effect occurs comprising a succession of alternating phases of vasodilatation and vasoconstriction. This without doubt also produces a draining effect and above all elasticisation of the microcirculation and its modulation around a main carrying event that causes its average variation.

In a series of experiments conducted after those described above, this type of vasoactive ATMC stimulation was associated with a vasodilative or vasoconstrictive stimulus. If the ATMC stimulus is accompanied by a vasodilative carrying stimulus, for example thermal heating stimulation, as in the case illustrated in FIG. 14, this association substantially enhances vasodilatation and the dose/response ratio.

On the other hand, if the ATMC stimulus is accompanied by a vasoconstrictive carrying stimulus, such as for example thermal cooling stimulation, this association substantially enhances vasoconstriction.

In this case Lorenz™ stimulation by means of the ATMC sequence creates effective neuromodulation that is able to amplify the excitation phenomena of the primary and secondary neuroceptors. Consequently, it is possible to use the ATMC vasoactive sequence also in combination with hyperthermia and cryotherapy treatments to enhance the effects of the latter.

In this way localised neoplasms and solid tumours can be treated by the combination of temperature effects with vasoactive effects.

If cryotherapy is combined with the vasoactive ATMC sequence the vasoconstrictive effects are increased, thereby producing localised hypoxia in a tumour mass, with consequent necrosis of the latter.

Similarly, by combining the vasoactive ATMC sequence with a hyperthermic therapy important vasodilatation is obtained that amplifies the necrotizing effect of the hyperthermia on a tumour mass.

In conclusion, it can certainly be stated that the Lorenz Therapy™ stimulation sequences induce reproducible and constant neurophysiological responses; the ATMC and DCTR sequences are able to stimulate different functional contingents, including the striated muscle, the smooth muscle and the mixed peripheral nerve.

The stimulation sequences are assembled on three fundamental parameters: the width of the stimulus, the frequency of the stimulus and the time wherein different combinations of width/frequency follow. The general operating model reflects the digital-analogue transmission that occurs in nervous transmission.

1-7. (canceled)

8. The combination of:
an electrostimulating apparatus for applying electrical stimuli to biological tissues; and
a device for exchanging heat with said biological tissues.

9. The combination according to claim 8, wherein said device for exchanging heat comprises a device for heating said biological tissues.

10. The combination according to claim 8, wherein said device for exchanging heat comprises a device for cooling said biological tissues.

11. The combination according to claim 9, wherein said device for exchanging heat comprises a device for cooling said biological tissues.

12. The combination according to claim 8, wherein said device for exchanging heat comprises a device for controlling the temperature of said biological tissues.

13. The combination according to claim 9, wherein said device for exchanging heat comprises a device for controlling the temperature of said biological tissues.

14. The combination according to claim 10, wherein said device for exchanging heat comprises a device for controlling the temperature of said biological tissues.

15. The combination according to claim 11, wherein said device for exchanging heat comprises a device for controlling the temperature of said biological tissues.

16. An electrostimulating apparatus that generates a relaxing sequence suitable for stimulating striated muscle fibre, based on three fundamental parameters: the width of the electric stimulation, the frequency of said stimulation and the intervals of time wherein a plurality of width/frequency combinations follows.

17. An electrostimulating apparatus that generates a vasoactive sequence of activation of the microcirculation suitable for stimulating the smooth muscle fibre and the postsynaptic neuroceptors, based on three fundamental parameters: the width of the electric stimulation, the frequency of said stimulation and the time wherein a plurality of combinations of width/frequency follow.
